(19) United States
(12) Patent Application Publication
(10) Pub. No.: US 2014/0128476 A1
(43) Pub. Date: May 8, 2014

(54) Shape memory curable spinal implants

(71) Applicant: Boo Holdings, LLC, Palm Beach Gardens, FL (US)

(72) Inventor: Charles S. Theofilos, Palm Beach Gardens, FL (US)

(73) Assignee: Boo Holdings, LLC, Palm Beach Gardens, FL (US)

(21) Appl. No.: 14/070,016

(22) Filed: Nov. 1, 2013

Related U.S. Application Data
(60) Provisional application No. 61/721,753, filed on Nov. 2, 2012.

(51) Int. Cl. A61L 31/14; A61L 31/16; A61F 2/44

(52) U.S. Cl. CPC A61L 31/14; A61F 2/44; A61L 31/16
USPC 514/769; 623/17.11; 523/113; 525/122; 525/123; 525/154; 525/231; 514/772.4

(57) ABSTRACT

An implant having shape forming and hardening functionality is described. The implant is formed from shape memory materials. The implant is formed from a shape memory alloy, shape memory polymer, or combinations thereof, to provide them with the ability to be compressed into a shape that facilitates minimally invasive insertion. Subsequent to such insertion, the shape memory materials provide the implant with the ability to regain its original shape and size. Subsequently, the implant becomes rigid, thereby providing strength, structural integrity and support.

### PRIORITY CLAIM

In accordance with 37 C.F.R. 1.76, a claim of priority is included in an Application Data Sheet filed concurrently herewith. Accordingly, the present invention claims priority to U.S. Provisional Patent Application No. 61/721,753, entitled “SHAPE MEMORY CURABLE SPINAL IMPLANTS”, filed Nov. 2, 2012. The contents of which the above referenced application is incorporated herein by reference.

### FIELD OF THE INVENTION

This invention generally relates to spinal implants formed from a shape memory material, and particularly to implants capable of being reduced in size for minimally invasive insertion, subsequently returned to a previously memorized enlarged form, and then cured in vivo to result in a structurally stable implant.

### BACKGROUND OF THE INVENTION

It is well known to utilize prosthetic implants to aid in the healing and correction of orthopedic injuries and ailments. Bone fractures and other orthopedic injuries, whether the result of trauma or as an aftermath of a surgical procedure, take a substantial time to heal, during which time the bony structure is unable to support physiologic loads.

Orthopedic implant devices are well known which are designed to provide structural support to a patient's spine or other bone or joint. Such implants can be rigid to prevent motion between the bony portions, or can be flexible to allow at least limited motion between the bony portions while providing a stabilizing effect. As used herein, bony portions can be portions of bone that are separated by one or more joints, fractures, breaks, or other space. Implants can be positioned, for example, for use in rigid posterior spinal fixation systems, such as rods, plates, tethers and staples; for use in interbody spinal fusion or corpectomy; for use in dynamic spinal stabilization; or for rigid or dynamic stabilization of other bones or skeletal joints.

Although it is well understood that stabilization of adjacent bony portions can be completed with an implant positioned between the bony portions and/or an implant positioned along the bony portions, the ability to access these areas in a minimally invasive way, while delivering a sufficiently robust and particularly conformed implant to the site requiring stabilization, is often problematic. Therefore, there is a need for additional contributions in this area of technology.

### DESCRIPTION OF THE PRIOR ART

U.S. Pat. No. 7,771,476 to Justis et al, is directed toward orthopedic implant devices which are non-rigid, i.e., flexible and/or malleable, in a first form for insertion into a desired in vivo site, and then transformable into a rigid, or hardened, form for providing a load-bearing function or providing other structural and/or mechanical function after implant. The device includes a biocompatible sheath and a curable material sealed within the sheath. The curable material is provided in a first form that provides flexibility to the device and is structured to rigidize in a second form after insertion to an in vivo location as a result of application of a cure-initiating energy to the material prior to insertion. Related methods and kits are also provided.

WO 2002/34310 relates to a self-forming implant for treatment of muscular skeletal diseases and general orthopedic defects. The implant is formed of a shape memory polymer (SMP) and fabricated into a pre-selected configuration.

### SUMMARY OF THE INVENTION

The present invention is directed towards orthopedic implants, which are formed from shape memory materials. The improvement comprises initially forming these implants from a shape memory alloy, shape memory polymer, or combinations thereof, to provide these implants with the ability to be compressed into a shape which facilitates minimally invasive insertion. Subsequent to such insertion, the shape memory materials provide the implant with the ability to regain its original shape and size. Subsequently, with the addition of a specific form of energy, the implant becomes rigid, so that it is capable of providing the structural integrity and support normally derived from a non-deformable implant.

Accordingly, it is a primary objective of the instant invention to teach orthopedic, e.g. spinal implants, formed from a combination of shape memory materials capable of being formed into well-defined orthopedic implants while retaining the ability to be initially deformed into a shape to facilitate minimally invasive insertion, followed by transformation into a rigid structure subsequent to regaining its originally defined size and shape, and ultimately treated to become rigid in vivo, whereby structural integrity and strength are achieved.

It is a further objective of the instant invention to teach kits including implants in accordance with the instant invention in combination with the requisite energy supplying devices to enable reformation and rigidizing of the implant subsequent to insertion.

Other objects and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE FIGURES

FIG. 1A is a schematic illustration of an illustrative example of a shape memory, curable implant device;

FIG. 1B is a perspective view of an illustrative embodiment of the implant device in accordance with the present invention, shown as a spinal implant device;

FIG. 2 is an alternative embodiment of a shape memory, curable implant device;

FIG. 3 illustrates the conversion of the that shape memory, curable implant device from the compresses state to a non-compressed state or original configuration;

FIG. 4 is illustrative of a minimally invasive insertion technique for a compressed implant which subsequently expands;

FIG. 5 illustrates an embodiment of the shape memory, curable implant device, in the decompressed state, inserted between two vertebral bodies;

FIG. 6 illustrates the shape memory, curable implant device illustrated in FIG. 5 assuming its original shape;

FIG. 7 illustrates the shape memory, curable implant device illustrated in FIG. 6 assuming its final position and being cured for strength.

### DETAILED DESCRIPTION OF THE INVENTION

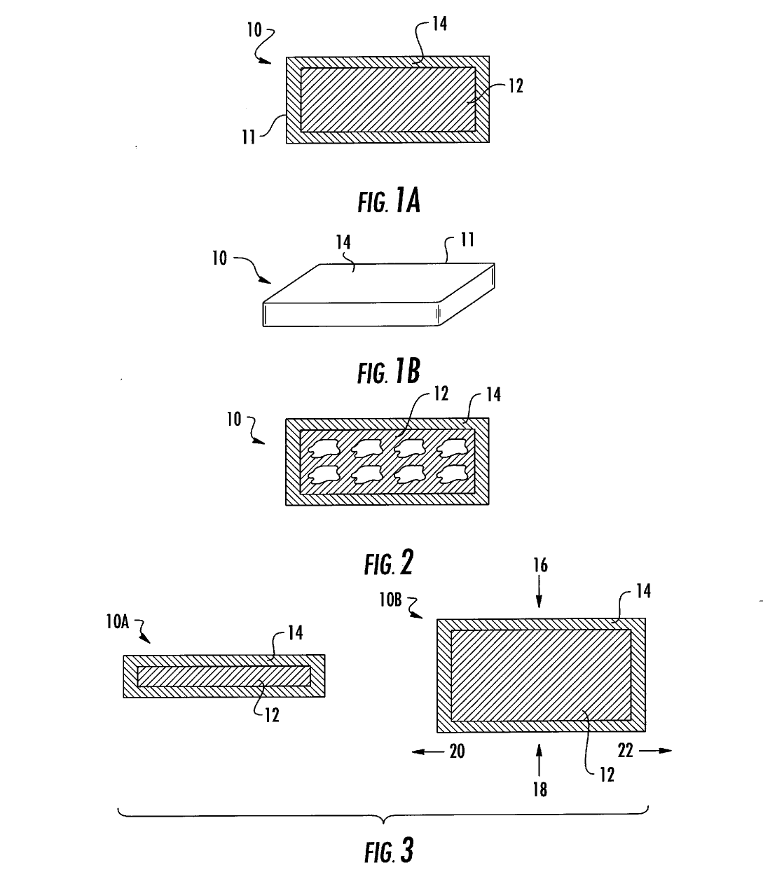

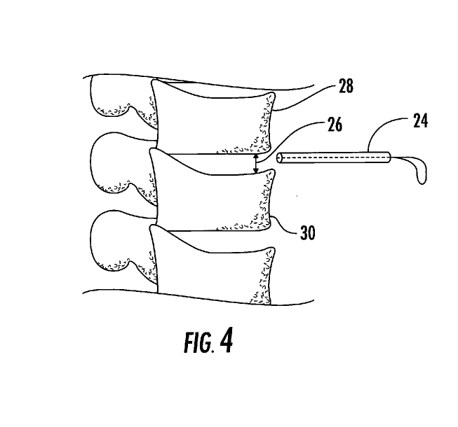

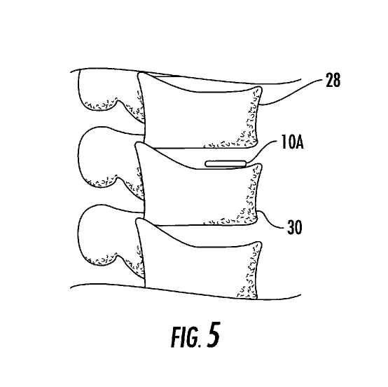

Implants in accordance with the present invention are capable of being reduced in size for minimally invasive insertion and subsequently returned to a previously memorized enlarged form, subsequent to which they may be exposed to a form of energy or chemical initiator to effectuate curing or hardening in vivo, thereby resulting in a structurally stable implant. Referring to FIGS. 1A and 1B, an illustrative embodiment of an implant device, generally referred to as 10, is shown. As shown in FIG. 1B, the implant device 10 is illustrated as a spinal implant device insertable between vertebral bodies. However, the implant device 10 in accordance with the present invention is not limited to spinal procedures. The implant device 10 comprises a main body 11 comprised of at lest two component materials, a first component material 12 and a second competent material 14. Both the first component material 12 and the second component material 14 have different chemical or physical characteristics so as to provide an implant device that provides 1) shape memory features/functionality, and 2) is curable. Accordingly, the first component 12 may be constructed of a material that provides shape memory to allow the spinal implant to traverse, or change configurations form a compressed state, 10A, to a decompressed, or larger, original shape 10B, see FIG. 3. Traversal between the two states can be accomplished by a traversing force or energy as described later.

While the spinal implant 10B is strong enough to hold its shape, insertion between vertebral bodies creates forces or pressures, see arrows 16 or 18, which may also produce additional side forces 20 and 22 the spinal implant must withstand to be effective. To provide an implant that can withstand such forces, the second component is constructed of a material that can be hardened by curing using curing forces, energy sources or mechanisms as described later. Once hardened, the implant device 10 provides one or more load bearing surfaces. Both the first component material 12 and the second component material 14 must function via different means. For example, if the first material requires heat to traverse between the compressed state and the decompressed, original state, the second component must be made of a material that can undergo the curing process using non-thermal means, such as ultraviolet radiation or blue light.

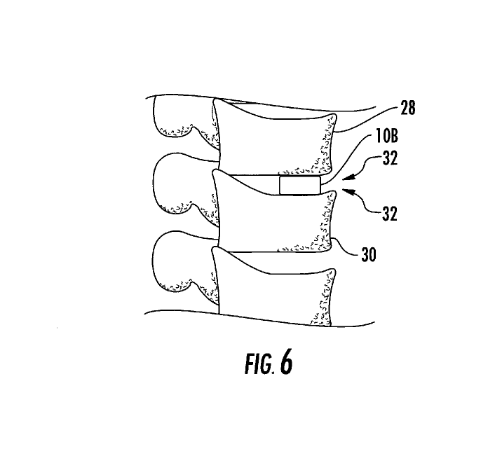

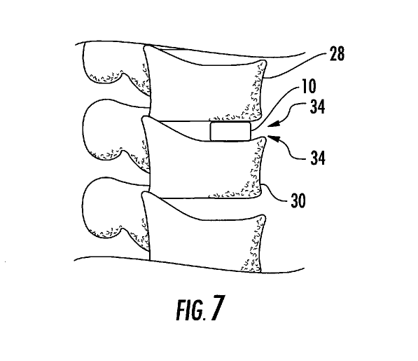

FIGS. 4-7 are illustrative of a minimally invasive insertion technique for inserting an implant device 10 in a compressed implant which subsequently expands into a mammal, preferably a human. As shown, an insertion tube 24 has been positioned at the intervertebral space 26 between two vertebral bodies 28 and 30. The compressed implant 10A is inserted within the intervertebral space 26, see FIG. 5. Once in position, the compressed implant 10A is expanded, see 10B of FIG. 6, using for example a thermal energy, see arrows 32. Once in the expanded state, the implant 10 is strengthen using a different energy source, see arrows 34, FIG. 7.

It is contemplated that the reformation and subsequent hardening or curing comprises two separate and distinct steps. As such, there is no pre-determined “working time” post insertion. Rather, the shape memory material is capable of first being exposed to a form of energy which will initiate transformation from a first compressed state, to a second original state, wherein it takes on the initial form and dimension which it had prior to compression.

Subsequently, the surgeon is able to discern if the selection of implant is acceptable in terms of size and orientation. If not, the implant may be recompressed and removed. If all is in order, then the surgeon can initiate the hardening or curing step to form a rigid implant in vivo.

In certain embodiments the device may contain precursors that are polymerized and/or cross-linked by a chemical reaction. At this time the device can provide a load-bearing function as well as other structural and/or mechanical function. In certain embodiments, the device may also continue initiators or catalysts.

In accordance with the present invention it is understood that the terms “hardenable,” “cured” or “curable” are used interchangeably herein, and are intended to refer to any material that can be stably stored for an extended period of time in a first, malleable or flexible form without loss of flexibility, and be transformed to a second, hardened form after application of an initiating energy thereto. No specific mechanism of hardening is preferred, and it should be understood that any mode of shape memory material transformation is contemplated herein.

As will be understood by those of skill in the art, a variety of hardening mechanisms can be utilized, depending upon material selection, including for example, curing that is initiated by ultraviolet radiation, visible light, blue light, infrared radiation, radio frequency radiation, x-ray radiation, gamma radiation or other wavelength of electromagnetic energy, catalyst-initiated polymerization, thermally-initiated polymerization, electrically-initiated polymerization, mechanically-initiated polymerization, curing initiated by electron beam radiation, and the like.

It is further understood that the material from which the implant 10 is formed can be a single material or a combination of materials selected so as to provide the requisite ability to be compressed ex vivo, inserted via a minimally invasive technique, decompressed in vivo, and subsequently rigidized in a step subsequent to the decompression step, in order to provide the surgeon with an ability to test the propriety of the implant prior to hardening thereof. The implant 10 may be constructed of a single unit comprising at least two component materials. Alternatively, the implant 10 may comprise of a first component, i.e. component material 12, and a second component, i.e. component material 14, that is coated or secured to the first component material 12.

Activatable polymeric materials which are useful in the invention include thioisocyanates, aldehydes, isocyanates, divinyl compounds, epoxides or acrylates. In addition to the aforementioned, photoactivatable crosslinkable groups such as succinimidyl azido salicylate, succinimidyl-azidobenzoate, succinimidyl dithio acetate, azidoiodobenzene, fluoro nitrophenylazide, salicylate azides, benzophenone-maleimide, and the like may be used as photoactivatable crosslinking reagents. The material may also consist of a thin coating, e.g. overlying a shape memory alloy, which can be activated by external forces such as laser, radio frequency, ultrasound or the like, with the same hardening result taking place.

In various embodiments contemplated in accordance with the present invention, shaped memory materials constituting shape memory polymers, shape memory alloys, or a material derived from a combination thereof, with or without osteogenic additives, are useful.

The implant 10 may be formed of a shape memory polymer (SMP) and fabricated into a pre-selected configuration. Fabrication of the implant using a shape memory polymeric material may impart certain novel characteristics to the implant.

The SMP implant can be molded into a desired configuration. Subsequently, when the implant is heated above a deformation temperature (Td)—which is usually equivalent to the glass transition temperature (Tg) of the polymeric material, the SMP becomes elastic. The implant can then be deformed to a wide variety of configurations by applying pressure or forcing it into a mold. Cooling the deformed implant below Td fixes it in its deformed state.

The deformed implant will then retain the deformed configuration until it is heated above Td. When the implant is reheated above Td, the SMP again becomes elastic; and in the absence of any applied pressure, the implant automatically reverts to its original configuration. This process can be repeated any number of times without detrimental effect on the SMP or the implant itself.

In an alternative embodiment the implant 10 is in the form of a deformable body for a bone structure formed of a shape memory polymeric material. The body is provided in a first configuration and is capable of deforming to a second configuration upon application of selected stimuli to conform to a portion of the bone structure.

The implant 10 can be provided with cavities, which can serve as depots for biological material such as an osteogenic material to promote bone fusion. In other preferred embodiments, the implant is deformable at a deformation temperature selected to be at body temperature and below.

In a further embodiment the invention provides an orthopedic implant for two or more adjacent bone portions, e.g. for insertion between vertebral bodies. The body is provided in a first configuration and adapted to bear against adjacent bone portions, wherein the body deforms upon application of selected stimuli to a second configuration to apply a force to the adjacent bone portions.

The shaped memory polymeric material can be selected from a wide variety of polymers, including biodegradable and non-biodegradable polymers. In preferred embodiments, the shape memory polymeric material is formed from oligomers, homopolymers, copolymers, and polymer blends that include polymerized monomers derived from l, d, or d/l lactide (lactic acid); glycolide (glycolic acid); ethers; olefins, such as ethylene, propylene, butene-1, pentene-1, hexene-1,4-methylpentene-1, styrene, norbornene and the like; butadiene; polyfunctional monomers such as acrylate, methacrylate, methyl methacrylate; esters, for example, caprolactone, and mixtures of these monomeric repeating units.

Copolymers may include random copolymers, graft copolymers, block copolymers, radial block, diblock, triblock copolymers, alternating copolymers, and periodic copolymers. Polymer blends are intended to include polymer alloys, semi-interpenetrating polymer networks (SIPN) and interpenetrating polymer networks (IPN).

More specifically, the polymers may be selected from homopolymers, copolymers, polymer blends, and oligomers of d, l, d/l, polylactide, polyglycolide, poly (lactide-co-glycolide), poly(β-hydroxy butyrate); poly((β-hydroxy butyrate-co-hydroxyvalerate), poly(trimethylene carbonate) polyurethane, poly(ethylene-co-vinyl acetate) (EVA), poly (ethylene-co-propylene) (EPR), poly(ethylene-co-propylene-co-diene) a ter-polymer (EPDM), poly(c-caprolactone), poly-imino carbonates, polyanhydrides, copolymers of ethylene and propylene and/or other α-olefins, or copolymers of these olefins. Among them, various types of polyethylene, such as low-density polyethylene, linear low-density polyethylene, medium-density polyethylene and high-density polyethylene, and polypropylene are preferable.

Exemplary polymers are described in U.S. Pat. No. 4,950,258, the entire disclosure of which is incorporated by reference herein.

The polymer composition of the present invention may further contain thermoplastic resins and/or thermoplastic elastomers to improve their stiffness, moldability and formability. In addition, the shape-memory molded implant may additionally include additives such as coloring agents, stabilizers, fillers, and the like, in an amount such as will not alter the desired shape-memory effect, biocompatibility and/or biodegradability properties of the molded implants.

The polymer is characterized in that it will attempt to assume its memory condition by activation of a polymer transition. Activation can occur by absorption of heat by the polymer, absorption of liquid by the polymer, or a change in pH in the liquid in contact with the polymer. The polymer may be formulated to be responsive to absorption of a liquid by incorporating in the polymer a hydrophilic material, such an n-vinyl pyrrolidone. Incorporation of a material such as methacrylic acid or acrylic acid into the polymer results in a polymer having a transition that is sensitive to pH. The polymer transition may be a thermally activated transition, where upon absorption of heat the polymer undergoes a glass transition or a crystalline melting point.

The osteogenic materials used in this invention preferably comprise a therapeutically effective amount of a bone inductive factor such as a bone morphogenic protein in a pharmaceutically acceptable carrier. Examples of factors include recombinant human bone morphogenic proteins (rhBMPs) rhBMP-2, rhBMP-4 and heterodimers thereof. However, any bone morphogenic protein is contemplated including bone morphogenic proteins designated as BMP-1 through BMP-13, which are available from Genetics Institute, Inc., Cambridge, Mass. All osteo-inductive factors are contemplated whether obtained as above or isolated from bone, inclusive of both allograft and autograft.

Shape memory alloys can also be useful, either as an alternative to the shape memory polymers or in combination therewith. Nitinol is a commonly-used Ti—Ni alloy with shape memory behavior that is used in essentially all types of medical device applications. Additional shape memory alloys are those based on Ti, Zr, Si, B, Be, Cr, Nb and Co in a composition in which at least one of these elements exists in a range of between 40 weight percent and greater than 99 weight percent. Other examples include alloys with at least 80 weight percent Ti with the addition of Al, V, Fe and/or Nb, and a 99 weight percent Ti alloy. Other examples of shape memory alloys include those described in U.S. Pat. Nos. 4,665,906 and 5,067,957 which describe medical devices and methods of installation using a non-specific shape memory alloy which displays stress induced martinsitic behavior, versus an activation temperature.

In one illustrative embodiment, the implant 10 is formed from a foamed shape memory alloy, having a degree of porosity (see pores 36, FIG. 2) such that shape memory polymers and osteogenic materials can be inserted therein. This embodiment provides a single implant which utilizes both shape memory polymer and alloy to provide unique characteristics.

When provided in the form of a kit, various elements may comprise the kit. While all are not necessary, it is contemplated that the components of the kit may include one or more of alternative types and sizes of implant 10, the requisite insertion tools therefore, means for decompressing, means for recompressing and means for curing the implant.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. An implant device for implanting within a region of a mammal comprising at least one component material which has shape memory functionality and at least one component material adapted to be curable.

2. The implant device for implanting within a region of a mammal according to claim 1 wherein said at least one component material having shape memory functionality is traversable between a compressed configuration and a non-compressed configuration.

3. The implant device for implanting within a region of a mammal according to claim 1 wherein said at least one component material adapted to be curable provides at least one load bearing surface upon activation by a curing source.

4. The implant device for implanting within a region of a mammal according to claim 1 wherein said at least one component material which has shape memory functionality is formed from oligomers, homopolymers, copolymers, or polymer blends.

5. The implant device for implanting within a region of a mammal according to claim 1 wherein said at least one component material which has shape memory functionality is a shape memory allow, a shape memory polymer, or combinations thereof.

6. The implant device for implanting within a region of a mammal according to claim 5 wherein said shape memory alloy is Nitinol.

7. The implant device for implanting within a region of a mammal according to claim 5 wherein said shape memory alloy consists of a coating.

8. The implant device for implanting within a region of a mammal according to claim 1 wherein said at least one component material adapted to be curable is coated onto at least one portion of said implant having at least one component material which has shape memory functionality.

9. The implant device for implanting within a region of a mammal according to claim 1 further including thermoplastic resins, thermoplastic elastomers, or combinations thereof.

10. The implant device for implanting within a region of a mammal according to claim 1 wherein said device further includes one or more additives.

11. The implant device for implanting within a region of a mammal according to claim 10 wherein said additives include coloring agents, stabilizers, fillers, or combinations thereof.

12. The implant device for implanting within a region of a mammal according to claim 1 wherein said at least one component material which has shape memory functionality is formed from a foamed shape memory alloy.

13. The implant device for implanting within a region of a mammal according to claim 1 wherein said device further includes osteogenic materials.

14. The implant device for implanting within a region of a mammal according to claim 12 wherein said device further includes osteogenic materials

15. The implant device for implanting within a region of a mammal according to claim 1 wherein said first component comprises of a material which requires a transformation source for traversing between the compressed state and the decompressed and said second component material requires a different mechanism of curing than that used for transformation.

16. The implant device for implanting within a region of a mammal according to claim 1 wherein said first component requires a thermal mechanism to traverse between a compressed state and decompressed state, and said second material comprises of a material that can undergo the curing process using a non-thermal mechanism.
